(19) United States
(12) Patent Application Publication
(10) Pub. No.: US 2025/0058063 A1
(43) Pub. Date: Feb. 20, 2025

(54) DRY POWDER FORMULATIONS FILLED IN AN INHALER WITH IMPROVED RESISTANCE TO HUMIDITY

(71) Applicant: CHIESI FARMACEUTICI S.P.A., Parma (IT)

(72) Inventors: Marco DI CASTRI, PARMA (IT); Giuseppe Antonio MULAS, PARMA (IT); Sara BOTTINI, PARMA (IT); Alan TWEEDIE, PARMA (IT); Daniel David HIGGINS, PARMA (IT); Odysseas Michail VARVOUNIS, PARMA (IT); Ralph Donald Quentin COLLINGS, PARMA (IT); Benjamin Michael RICHARDS, PARMA (IT); Paul Graham HAYTON, PARMA (IT)

(21) Appl. No.: 18/721,497

(22) PCT Filed: Dec. 19, 2022
(86) PCT No.: PCT/EP2022/086768

§ 371 Date: Jun. 18, 2024

(30) Foreign Application Priority Data
Dec. 21, 2021 (EP) 21216252.3

(51) Int. Cl. A61M 15/00; A61K 9/00; A61K 9/14; A61K 31/44

(52) U.S. Cl. CPC A61M 15/0065; A61K 9/0075; A61K 9/145; A61K 31/44; A61M 15/0026; A61M 2202/062; A61M 2202/064; A61M 2205/0216; A61M 2205/7527

(57) ABSTRACT

A drug product includes a multidose dry powder inhalation device. The device has a medicament chamber and a desiccant chamber adjacent to the medicament chamber, and a pharmaceutical composition therein. The pharmaceutical composition includes a pharmaceutically acceptable salt of tanimilast. The desiccant chamber is filled with molecular sieves.

### FIELD OF THE INVENTION

The invention generally relates to inhalation drug products and methods of manufacturing the same.

### BACKGROUND OF THE INVENTION

Inhalers are hand-held portable devices that deliver medication directly to the lungs. One class of inhalers is passive dry powder inhalers (“DPI”). A passive DPI is a patient driven device wherein the action of breathing in through the device draws the powder formulation of a medicament into the respiratory tract. DPIs are well recognized as devices for drug delivery to the lung for treatment of pulmonary and systemic diseases.

They can generally be divided in: i) single-dose (unit-dose) inhalers, for the administration of an individual dose of the active ingredient/s contained in capsule or blister loaded into the device and punctured by the patient immediately before use; ii) pre-metered multi-dose inhalers containing a series of blisters or capsules with the active ingredient/s formulation or iii) reservoir inhalers containing a larger amount of the powder formulation of active ingredient/s, corresponding to multiple doses sufficient for longer treatment cycles, which is metered from a storage unit just before inhalation.

A formulation for DPI is commonly a powder blend of active ingredients and a bulk solid pharmacologically inert, physiologically acceptable diluent, such as lactose. The inhaled particle size of the active ingredients should be optimized to deliver the drug deep into the lung to achieve efficacy. This efficacious particle size typically lies between 1-6 micron whereas particles larger than this, 6-10 micron, tend to be deposited in the upper airways without reaching the site of action. It is well known that stability of the powder as well as the aerosol performances could be affected by environmental conditions, humidity in particular.

Therefore, it is desirable to control humidity within a DPI device.

In the art, usually silica gel has been used, see for example EP079066. However, its capacity is relatively low and it is not able to maintain the internal humidity stable (Lehto V P and Lankinen T, Int. J. Pharm. 275, 155, 2004).

A different desiccant was disclosed in WO 2008/040841 wherein the desiccant system comprises a salt such as magnesium chloride or potassium acetate.

Alternative approaches to control the moisture absorption by dry powder products have been shown in US 2008/0063719 and WO 2012/028662.

Notwithstanding any potential progress that has been made regarding controlling humidity within the DPI device, there is still the need of more efficacious systems.

Furthermore, there is the need of more efficacious systems to protect from humidity, advantageously when the drug-containing powder formulation is intended for being stored in sub-tropical and tropical countries.

The compound of formula (I),

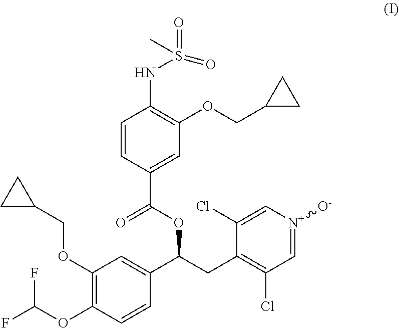 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

also named Tanimilast or CHF6001 or CHF-6001, with INN (3,5-dichloro-4-[(2S)-2-[3-(cyclopropylmethoxy)-4-(difluoromethoxy)phenyl]-2-{[3-(cyclopropylmethoxy)-4-(methanesulfonamido)benzoyl]oxy}ethyl]pyridine1-oxide), is an highly potent and selective PDE4 inhibitor with robust anti-inflammatory activity, currently under clinical development.

Compound of formula (I) has been disclosed in prior art documents in the name of Chiesi: WO 2009/018909 directed to its general formula, methods of preparation, compositions and therapeutic use; WO 2010/089107 specifically directed to sulphonamido derivatives as (−) enantiomers, including compound of formula (I), methods of preparation, compositions and therapeutic use; WO 2012/016889 directed to dry powder formulations comprising the compound of formula (I); WO 2015/059050 directed to crystalline form of the compound of formula (I) characterized by specific XRPD peaks and the process for obtaining it.

### SUMMARY OF THE INVENTION

In a first aspect, the invention provides a drug product comprising a multidose dry powder inhalation device, in turn, comprising a medicament chamber and a desiccant chamber adjacent to the medicament chamber, said device having a pharmaceutical composition present therein, the pharmaceutical composition comprising tanimilast, wherein the desiccant chamber is filled with molecular sieves.

In a particular embodiment, the multidose Dry Powder Inhaler comprises:
a casing (2) having a mouthpiece (4) and delimiting an inhalation channel connected to an opening (6) of the mouthpiece (4);
a container for storing a powdered medicament (medicament chamber) and placed in the casing (2);
a dispensing device placed in the casing (2) and configured to dispense unit doses of the powdered medicament from the container to the inhalation channel for inhalation through the mouthpiece (4);
a cover (3) engageable with the casing (2) to close the opening of the mouthpiece (4);
wherein the cover (3) comprises a sealing element (25) to further improve the resistance to humidity; and
whereby, when the cover (3) is engaged with the casing (2) and closes the mouthpiece (4), the main portion (26) of the sealing element (25) is coupled to the opening (6) to tight close said opening (6).

In another aspect, the invention provides a method for the treatment of a respiratory disorder. The method comprises administering to a patient by oral inhalation tanimilast, using a drug product as described herein in the first aspect.

In another aspect, the invention provides a process for manufacturing a drug product comprising a step of filling the medicament chamber of a multidose dry powder inhalation device with a pharmaceutical composition comprising tanimilast, and the desiccant chamber of said device with molecular sieves.

### Definitions

Unless otherwise specified, the compound of formula (I) of the present invention is intended to include also stereoisomers, tautomers or pharmaceutically acceptable salts, hydrates, addition complexes or solvates thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “racemate” or “racemic mixture” refers to a composition composed of equimolar quantities of two enantiomeric species, wherein the composition is devoid of optical activity.

The term “tautomer” refers to each of two or more isomers of a compound that exist together in equilibrium and are readily interchanged by migration of an atom or group within the molecule.

The “medicament chamber” is also defined in the art as “reservoir chamber”.

The term “micronized” refers to a substance having a size of few microns.

The term “coarse” refers to a substance having a size of one or few hundred microns.

In general terms, the particle size of particles is quantified by measuring a characteristic equivalent sphere diameter, known as volume diameter, by laser diffraction.

The particle size can also be quantified by measuring the mass diameter by means of suitable known instrument such as, for instance, the sieve analyser.

The volume diameter (VD) is related to the mass diameter (MD) by the density of the particles (assuming a size independent density for the particles).

In the present application, the particle size of the active ingredients and of fraction of fine particles is expressed in terms of volume diameter, while that of the coarse particles is expressed in terms of mass diameter.

The particles have a normal (Gaussian) distribution which is defined in terms of the volume or mass median diameter (VMD or MMD) which corresponds to the volume or mass diameter of 50 percent by weight of the particles, and, optionally, in terms of volume or mass diameter of 10% and 90% of the particles, respectively.

Another common approach to define the particle size distribution is to cite three values: i) the median diameter d(0.5) which is the diameter where 50% of the distribution is above and 50% is below; ii) d(0.9), where 90% of the distribution is below this value; iii) d(0.1), where 10% of the distribution is below this value.

The span is the width of the distribution based on the 10%, 50% and 90% quantile and is calculated according to the formula.

$$Span = \frac{D [ v , 0.9 ] - D [ v , 0.1 ]}{D [ v , 0.5 ]}$$

In general terms, particles having the same or a similar VMD or MMD can have a different particle size distribution, and in particular a different width of the Gaussian distribution as represented by the d(0.1) and d(0.9) values.

Upon aerosolisation, the particle size is expressed as mass aerodynamic diameter (MAD), while the particle size distribution is expressed in terms of mass median aerodynamic diameter (MMAD) and Geometric Standard Deviation (GSD). The MAD indicates the capability of the particles of being transported suspended in an air stream. The MMAD corresponds to the mass aerodynamic diameter of 50 percent by weight of the particles.

The term “hard pellets” refers to spherical or semispherical units whose core is made of coarse excipient particles.

The term “spheronisation” refers to the process of rounding off of the particles which occurs during the treatment.

The term “good flowability” refers to a formulation that is easy handled during the manufacturing process and is able to ensure an accurate and reproducible delivering of the therapeutically effective dose.

Flow characteristics can be evaluated by different tests such as angle of repose, Carr's index, Hausner ratio or flow rate through an orifice.

In the context of the present application the flow properties were tested by measuring the flow rate through an orifice according to the method described in the European Pharmacopeia (Eur. Ph.) 7.3, 7^{th} Edition.

The expression “good homogeneity” refers to a powder wherein, upon mixing, the uniformity of distribution of a component, expressed as coefficient of variation (CV) also known as relative standard deviation (RSD), is less than 5.0%. It is usually determined according to known methods, for instance by taking samples from different parts of the powder and testing the component by HPLC or other equivalent analytical methods.

The expression “respirable fraction” refers to an index of the percentage of active particles which would reach the lungs in a patient.

The respirable fraction is evaluated using a suitable in vitro apparatus such as Andersen Cascade Impactor (ACI), Multi Stage Liquid Impinger (MLSI) or Next Generation Impactor (NGI), according to procedures reported in common Pharmacopoeias, in particular in the European Pharmacopeia (Eur. Ph.) 7.3, 7^{th} Edition.

It is calculated by the percentage ratio of the fine particle mass (formerly fine particle dose) to the delivered dose.

The delivered dose is calculated from the cumulative deposition in the apparatus, while the fine particle mass is calculated from the deposition of particles having a diameter<5.0 micron.

In the context of the present application, the formulation is defined as extrafine formulation when it is able of delivering a fraction of particles having a particle size equal or less than 2.0 micron equal to or higher than 20%, preferably equal to or higher than 25%, more preferably equal to or higher than 30% and/or it is able of delivering a fraction of particles having a particle size equal or less than 1.0 micron equal to or higher than 10%.

The expression “physically stable in the device before use” refers to a formulation wherein the active particles do not substantially segregate and/or detach from the surface of the carrier particles both during manufacturing of the dry powder and in the delivery device before use. The tendency to segregate can be evaluated according to Staniforth et al. J. Pharm. Pharmacol. 34,700-706, 1982 and it is considered acceptable if the distribution of the active ingredient in the powder formulation after the test, expressed as relative standard deviation (RSD), does not change significantly with respect to that of the formulation before the test.

The expression “chemically stable” refers to a formulation that, upon storage, meets the requirements of the EMEA Guideline CPMP/QWP/122/02 referring to ‘Stability Testing of Existing Active Substances and Related Finished Products’.

The term “surface coating” refers to the covering of the surface of the carrier particles by forming a film of magnesium stearate around said particles. The thickness of the film has been estimated by X-ray photoelectron spectroscopy (XPS) to be approximately of less than 10 nm. The percentage of surface coating indicates the extent by which magnesium stearate coats the surface of all the carrier particles.

The term “prevention” means an approach for reducing the risk of onset of a disease.

The term “treatment” means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. The term can also mean prolonging survival as compared to expected survival if not receiving treatment.

According to the Global Initiative for Asthma (GINA), “severe persistent asthma” is defined as a form characterized by daily symptoms, frequent exacerbations, frequent nocturnal asthma symptoms, limitation of physical activities, forced expiratory volume in one second (FEV₁) equal to or less than 60% predicted and with a variability higher than 30%.

According to the Global initiative for chronic Obstructive Pulmonary Disease (GOLD) guidelines, “severe COPD” is a form characterized by a ratio between FEV₁ and the Forced Vital Capacity (FVC) lower than 0.7 and FEV₁ between 30% and 50% predicted. The very severe form is further characterized by chronic respiratory failure.

“Therapeutically effective dose” means the quantity of active ingredients administered at one time by inhalation upon actuation of the inhaler. Said dose may be delivered in one or more actuations, preferably one actuation (shot) of the inhaler.

“Actuation” refers to the release of active ingredients from the device by a single activation (e.g. mechanical or breath).

To check stability, studies of pharmaceutical drugs shall be performed under different conditions according to the climatic conditions of the country. According to ICH guidelines for stability studies the world climate is divided in five different zones:

| Zone | Type of Climate |
|---|---|
| Zone I | Temperate zone |
| Zone II | Mediterranean/subtropical zone |
| Zone III | Hot dry zone |
| Zone IVa | Hot humid/tropical zone |
| Zone IVb | Hot/higher humidity |

As used in this specification and the appended claims, the singular forms “a”, “an”, “the” and “one” include plural referents unless the content clearly dictates otherwise. Thus, for example, reference to “a salt” includes two or more such salts; reference to “a constituent” includes two or more such constituents and the like.

### FIGURES

FIG. 1 shows a 3D view of a powder inhaler according to the present invention in an open configuration.

FIG. 2 shows an enlarged view of a portion of the powder inhaler of FIG. 1.

FIG. 3 is a plot showing the results obtained as described in Example 1: A) cumulative weight increment (mg, ordinates) over time (days, abscissae) due to moisture absorption in an inhaler comprising molecular sieves in the desiccant chamber (PROPAGROUP, grey line) compared to an inhaler comprising silica gel (STD, black line); B) a differential weight (mg, ordinates) over time (days, abscissae) due to moisture absorption in an inhaler comprising molecular sieves in the desiccant chamber (dotted bar) compared to an inhaler comprising silica gel (slash lines bar). In B) the higher is the bar, the greater is the reduction of moisture absorption.

FIG. 4 is a chart showing a differential weight over time due to moisture absorption of inhalers incorporating the separate different embodiments of the invention compared to a standard inhaler according to WO 2016/000983. The higher is the bar, the greater is the reduction of moisture absorption.

FIG. 5 shows a 3D view of an internal element of the device according to WO 2004/012801 or WO 2016/000983, the metering member, or shuttle, of the inhaler shaped like a plate.

FIG. 6 shows a view of the elastomer island embodiment applied at a metering member of FIG. 5

FIG. 7A is an exploded 3D view of some internal components of the inhaler according to WO 2004/012801 or WO 2016/000983 including the gasket embodiment.

FIG. 7B shows a 3D view of one element of the powder inhaler from the view of FIG. 7A related to the gasket embodiment.

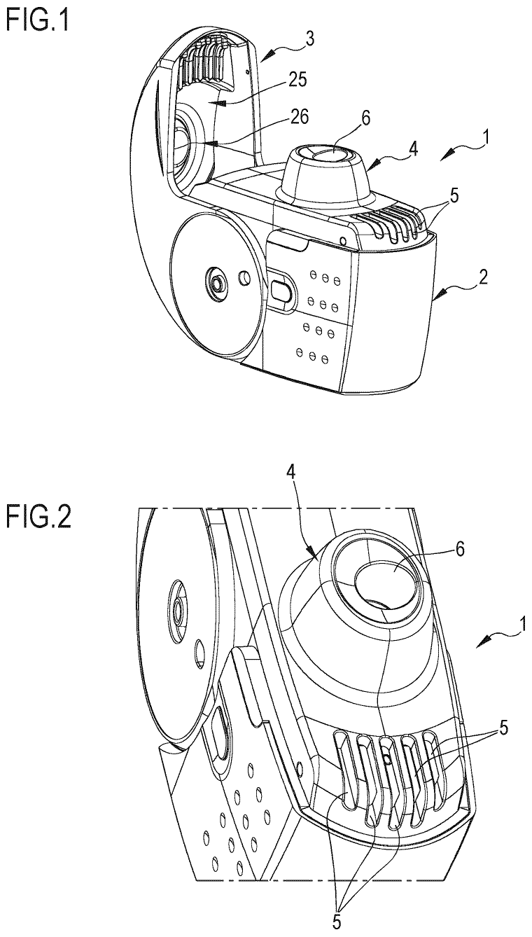

### DETAILED DESCRIPTION OF THE INVENTION

The invention will now be described with respect to the embodiments presented herein. Before describing the present invention in detail, it is to be understood that this invention is not limited to particularly exemplified structures, apparatus, systems, materials or methods, which as such may of course vary. Thus, although a number of apparatus, systems and methods similar or equivalent to those described herein can be used in the practice of the present invention, the preferred apparatus, systems and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

In a first aspect, the invention provides a drug product comprising a multidose dry powder inhalation device comprising a medicament chamber and a desiccant chamber adjacent to the medicament chamber, said device having a pharmaceutical composition present therein, the pharmaceutical composition comprising, as active ingredient, a pharmaceutically acceptable salt of tanimilast, wherein the desiccant chamber is filled with molecular sieves.

Said molecular sieves are made of a material with pores of uniform size and they could absorb small molecules such as water. Typically, they are made of alkaline salts of aluminosilicates, called zeolites, with pores having a diameter comprised from 2 to 50 angstrom, preferably from 3 to 20 angstrom. In a preferred embodiment, the diameter is 10 angstrom. Other suitable materials know in the art, such as aluminophosphates, porous glass or active carbon could advantageously be used. Artificial zeolites could also be used.

In a particular embodiment, the molecular sieves are contained in a Tyvek® bag able of being inserted in the desiccant chamber, Typically, the bag is inserted inside the desiccant chamber and after that the foil will be sealed to seal the desiccant chamber itself. In an alternative embodiment, the desiccant chamber is filled with the molecular sieves in form of a single tablet.

The amount of molecular sieves will depend on the geometry and the volume of the desiccant chamber. In one embodiment, the amount of molecular sieve per bag is 0.2-1.5 g.

Advantageously, the desiccant chamber and the medicament chamber are separated by a permeable membrane.

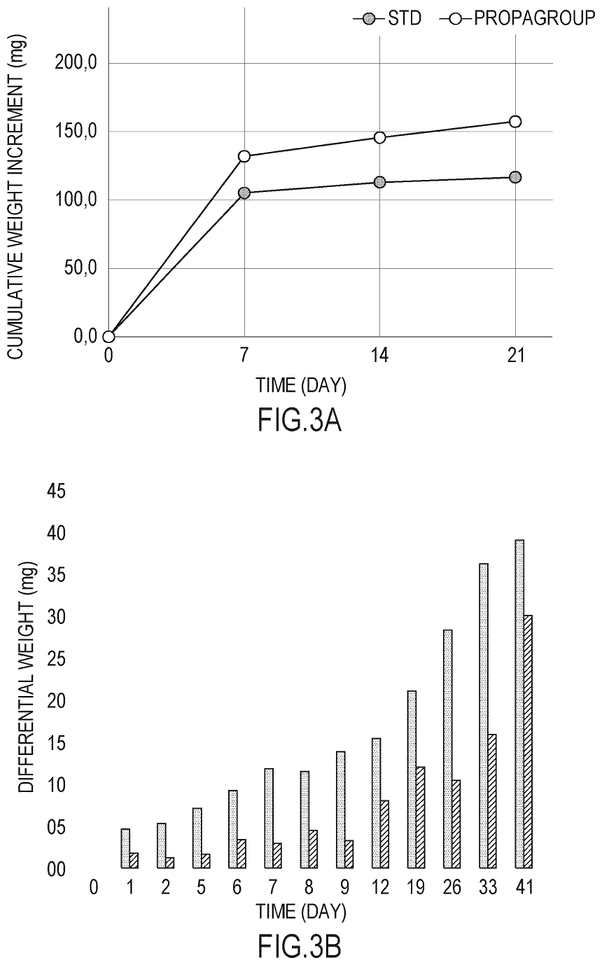

It has been found that molecular sieves are very efficient in increasing the speed of humidity extraction during the use of the inhaler. In fact, as reported in FIG. 3, molecular sieves absorb humidity much quicker than silica gel, allowing for a more dry environment of the desiccant chamber to be maintained.

Accordingly, the drug product is capable of exhibiting improved shelf life and more stable fine particle fraction as a result of the system of the invention.

In particular, the formulation filled in the device turned to be physically and chemically stable before use and during storage, while maintaining good homogeneity and flowability and a good respirable fraction over time.

It was also surprisingly found that said good properties are maintained when the drug product is exposed to temperature and relative humidity conditions typical of sub-tropical and tropical countries.

In addition to the medicament chamber and the desiccant chamber, the Dry Powder Inhaler (DPI) shall comprise a mouthpiece through which the user could inhale the powder medicament.

Advantageously, it further comprises a case with a lower shell and an integral cover, being pivotably or rotatably coupled to the lower shell.

The cover could be opened to reveal the mouthpiece.

In a preferred embodiment, the Dry Powder inhaler comprises:
a casing (2) having a mouthpiece (4) and delimiting an inhalation channel connected to an opening (6) of the mouthpiece (4);
a container for storing a powdered medicament and placed in the casing (2);
a dispensing device placed in the casing (2) and configured to dispense unit doses of the powdered medicament from the container to the inhalation channel for inhalation through the mouthpiece (4);
a cover (3) engageable with the casing (2) to close the mouthpiece (4);
wherein the cover (3) comprises a sealing element (25) to further improve the resistance to humidity; and
whereby, when the cover (3) is engaged with the casing (2) and closes the mouthpiece (4), the main portion (26) of the sealing element (25) is coupled to the opening (6) to tight close said opening (6).

The sealing element (25) is made of a soft or a medium soft material and is more deformable and flexible than the material of the mouthpiece.

Advantageously, the sealing element (25) has a hardness comprised between 10 Shore A and 60 Shore A, preferably comprised between 20 Shore A and 40 Shore A, more preferably comprised between 25 Shore A and 35 Shore A.

The Shore A Hardness Scale measures the hardness of flexible mold rubbers that range in hardness from very soft and flexible, to medium and somewhat flexible, to hard with almost no flexibility at all. In some embodiments, the sealing element (25) is made of silicone, e.g. PlatSil® FS-20 mixed with PlatSil® GEL.

Alternative materials such as thermoplastic elastomers could be used. Suitable TPE may be selected from those of medical-pharmaceutical grade belonging to classes of styrene block copolymers, thermoplastic polyolefin elastomers, and thermoplastic polyurethanes. Preferably silicone is used.

Said element could have a thickness variable between about 0.2 and 5 mm, preferably 1 and 2 mm, and a shape that mimic the external geometry of the mouthpiece, in order to fill the gaps of the mouthpiece itself and avoid that humidity could enter through those gaps.

Typically, the sealing element is over-molded to the cover (3) or press-fitted in the cover (3) and optionally glued to the cover (3) or mechanically connected to the cover (3).

Advantageously, the casing (2) has at least one air inlet (5) in fluid communication with the inhalation channel to allow air intake at least when the user draws from the mouthpiece (4); wherein, when the cover (3) is engaged with the casing (2) and closes the mouthpiece (4), the main portion (26) of the sealing element (25) is coupled to the at least one air inlet (5) to tight close said at least one air inlet (5).

Exemplary Dry Powder Inhalers (DPIs) with a cover engageable with the casing to close the mouthpiece suitable to host the sealing element and with a desiccant chamber suitable for being filled with the molecular sieves are reported in WO 2004/012801 and WO 2016/000983 to which the reader is referred for the drawings and numbering of the various parts, except for the numbering herein reported.

Advantageously, the cup of the medicament chamber of the DPI according to WO 2004/012801 or WO 2016/000983 is configured to deliver 180 doses rather than 120 doses, as it has been observed that the larger the cup, more mandatory is the request of having a more protective effect against the ingress of moisture.

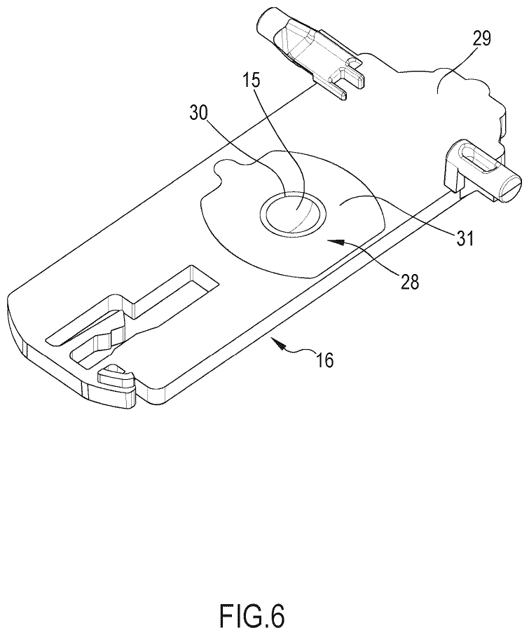

Typically, the dispensing device of the DPI according to WO 2004/012801 or WO 2016/000983 comprises a shuttle (FIG. 5; (16) of FIG. 6) having a dosing recess, wherein said shuttle is movable between a filling position, in which the dosing recess is in alignment with an opening of the container and faces said opening so as to be filled with a dose of the powdered medicament, and an inhalation position, in which the dosing recess is in alignment with the inhalation channel for enabling inhalation of the dose of the powdered medicament contained in the dosing recess through the mouthpiece.

According to a preferred embodiment, in order to further reduce the access of humidity, the DPI further comprises a sealing device operationally active at a coupling zone of the shuttle with the container when the shuttle is in the filling position, said coupling zone circumscribing the dosing recess and the opening.

In a more preferred embodiment (elastomer island, FIG. 6), the sealing device comprises a deformable portion (31) surrounding the dosing recess (15) and connecting the dosing part (30) to the main part (29), wherein the deformable portion is lesser stiff than the main part (29) along a direction perpendicular to a lying plane of the shuttle; wherein at least one spring is interposed between the casing (2) and the dosing part and is configured to push said dosing part against the opening of the container by deforming the deformable portion of the shuttle when the shuttle is in the filling position.

According to said more preferred embodiment, the shuttle could be shaped like a plate and the main part (29) could have a first wall thickness (t1), wherein the deformable portion has a second wall thickness (t2) smaller than the first wall thickness (t1), optionally wherein a ratio t2/t1 of the second wall thickness (t2) to the first wall thickness (t1) is smaller than 0.5, optionally smaller than 0.3.

Advantageously, the dosing part could comprise a peripheral stiffening rib surrounded by the deformable portion, wherein the deformable portion has an average width (wₐᵥ) and a ratio t2/wₐᵥ of the second wall thickness (t2) to the average width (wₐᵥ) is smaller than 0.3, optionally smaller than 0.2, optionally smaller than 0.1.

Typically, the main part (29), the deformable portion (31) and the dosing part (30) are made in a single piece, optionally of plastic, optionally of acrylonitrile butadiene styrene (ABS), Preferably, the deformable portion is made of or comprises an elastomeric material, optionally a medical grade butyl rubber.

The dosing part (30) could comprise at least one stiffening rib on a side opposite the dosing recess.

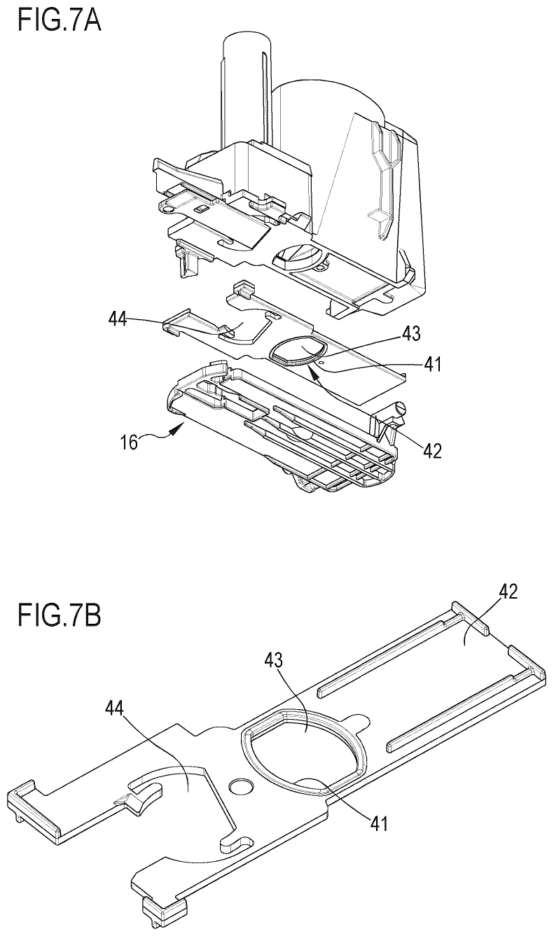

In an alternative embodiment (gasket, FIG. 7), the sealing device could comprise a gasket (41) placed at the coupling zone, at least when the shuttle is in the filling position.

In this embodiment, the Dry Powder Inhaler comprises a support plate (42) made of plastic, e.g. acetal resin. The support plate (42) is sandwiched between the container and the shuttle. The support plate (42) is anchored to the casing (2).

The support plate (42) has a through opening (43) and a through inhalation passage (44).

Advantageously, the gasket could be made of an elastomeric material, preferably selected from thermoplastic elastomers (TPEs) of medical-pharmaceutical grade or silicone, and the support plate is made of plastic, optionally acetal resin. Preferably, said gasket has a raised bead protruding towards the container and/or a raised bead protruding towards the shuttle.

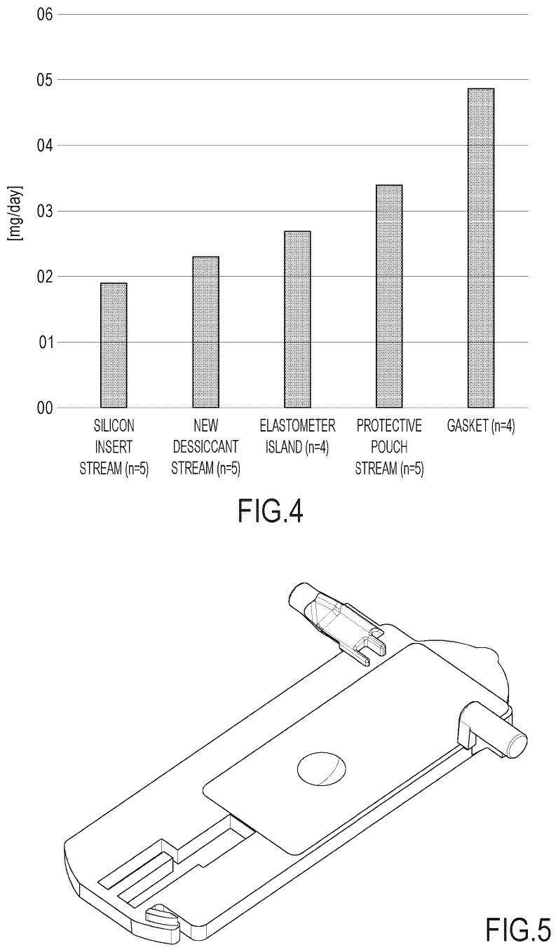

As it can be appreciated for FIG. 4, which shows a differential weight over time due to moisture absorption of inhalers incorporating separate different embodiments of the invention compared to a standard inhaler, both the elastomer island and the gasket are very effective in decreasing the absorption of moisture.

The pharmaceutical composition filling the medicament chamber of the multidose inhaler (DPI) shall be in form of dry powder. Advantageously, it comprises a fraction of fine excipients particles a), a fraction of coarse excipient particles b), and micronized particles of a pharmaceutically acceptable salt of tanimilast.

The fractions a) and b) are the “carrier” particles.

Advantageously, the fine and coarse excipient particles may consist of any pharmacologically inert, physiologically acceptable material or combination thereof; preferred excipients are those made of crystalline sugars, in particular lactose; the most preferred are those made of α-lactose monohydrate.

Preferably, the coarse excipient particles and the fine excipient particles are made of the same material and both consist of alpha-lactose monohydrate.

The coarse excipient particles of the fraction b) must have mass median diameter equal to or higher than 100 micron preferably equal to or greater than 125 micron, more preferably equal to or greater than 150 micron, even more preferably equal to or greater than 175 micron.

Advantageously, all the coarse particles have a mass diameter in the range 50-1000 micron, preferably comprised between 60 and 500 micron.

In certain embodiments of the invention, the mass diameter of said coarse particles might be comprised between 80 and 200 micron, preferably between 90 and 150 micron, while in another embodiment, the mass diameter might be comprised between 200 and 400 micron, preferably between 210 and 380 micron.

In a preferred embodiment of the invention, the mass diameter of the coarse particles is comprised between 210 and 360 micron.

In general, the person skilled in the art shall select the most appropriate size of the coarse excipient particles by sieving, using a proper classifier.

When the mass diameter of the coarse particles is comprised between 200 and 400 micron, the coarse excipient particles preferably have a relatively highly fissured surface, that is, on which there are clefts and valleys and other recessed regions, referred to herein collectively as fissures. The “relatively highly fissured” coarse particles can be defined in terms of fissure index or rugosity coefficient as described in WO 01/78695 and WO 01/78693, whose teachings are incorporated herein by reference, and they could be characterized according to the description therein reported. Advantageously, the fissure index of said coarse particles is of at least 1.25, preferably of at least 1.5, more preferably of at least 2.0, while the rugosity coefficient is of at least 1.01, preferably comprised between 1.02 and 1.3.

Advantageously, the ratio between the fraction of fine excipient particles a) and the fraction of coarse excipient particles b) could be comprised between 1:99 and 30:70 by weight, preferably between 10:90 and 20:80 by weight.

Advantageously, the fraction of fine excipient particles a) consists of particles of a physiologically acceptable excipient and particles of a suitable additive, wherein at least 90% of all the particles have a volume diameter lower than 15 micron, preferably lower than 12 micron.

The ratio between the excipient particles and the additive particles within the fraction a) may vary depending on the doses of the active ingredients.

The additive material may include a combination of one or more materials, and it may be selected from aminoacids such as leucine and isoleucine or surface active substances such as stearate salts.

In a preferred embodiment, the additive is magnesium stearate as, due to its hydrophobicity, it is capable of improving the moisture resistance of dry powder formulations for inhalation as disclosed in WO 00/28979.

Advantageously, when magnesium stearate is used, the fraction of fine particles a) is composed of 90 to 98% by weight of the excipient and 2 to 10% by weight of magnesium stearate. In a particular embodiment, the amount may be 98% of the excipient particles and 2% of magnesium stearate, by weight.

To further improve the resistance to humidity, it is preferred subjecting the mixture of the physiologically acceptable excipient with magnesium stearate to co-micronization in a mill as disclosed in WO 01/78693 rather than simply mixing them.

Typically, the particles are co-micronized starting from excipient particles having a mass diameter lesser than 250 micron and magnesium stearate particles having a mass diameter lesser than 35 micron using a jet mill, preferably in inert atmosphere, for example under nitrogen.

As an example, alpha-lactose monohydrate commercially available such as Meggle D 30 or Spherolac 100 (Meggle, Wasserburg, Germany) could be used as starting excipient.

Optionally, the fraction of fine excipient particles a) may be subjected to a conditioning step according to the conditions disclosed in the pending application n. WO 2011/131663.

Although the time of treatment will generally depend on the starting particle size of the excipient particles and the desired size reduction to be obtained, it is preferably performed for at least one hour, preferably for at least two hours, more preferably for four hours or more.

It has indeed been found that, the greater is the intimate contact of the magnesium particles with the fine excipient particles, the higher is the capability of magnesium stearate to protect the formulation from the detrimental effect of humidity.

Therefore, by performing co-micronization for rather long time, it may be possible reducing the amount of magnesium stearate to be used in the formulation, while maintaining a good resistance to humidity.

For instance, the ratio between the fine excipient particles and magnesium stearate particles may be preferably of 99/1 by weight, more preferably 99.5/0.5.

To determine the intimacy between the excipient particles and magnesium stearate in fine fraction a), the extent of coating could be determined.

Advantageously, magnesium stearate coats the surface of the excipient particles of fine fraction a) in such a way that the extent of the surface coating is higher than 20%, preferably higher than 50%, more preferably higher than 60%.

The extent to which the magnesium stearate coats the surface of the excipient particles may be determined by X-ray photoelectron spectroscopy (XPS), a well known tool for determining the extent as well as the uniformity of distribution of certain elements on the surface of other substances. In the XPS instrument, photons of a specific energy are used to excite the electronic states of atoms below the surface of the sample. Electrons ejected from the surface are energy filtered via a hemispherical analyser (HSA) before the intensity for a defined energy is recorded by a detector. Since core level electrons in solid-state atoms are quantized, the resulting energy spectra exhibit resonance peaks characteristic of the electronic structure for atoms at the sample surface.

Typically XPS measurements are taken on an Axis-Ultra instrument available from Kratos Analytical (Manchester, UK) using monochromated Al Kα radiation (1486.6 eV) operated at 15 mA emission current and 10 kV anode potential (150 W). A low energy electron flood gun is used to compensate for insulator charging. Survey scans, from which quantification of the detected elements are obtained, are acquired with analyser pass energy of 160 eV and a 1 eV step size. High-resolution scans of the C 1s, O 1s, Mg 2s, N 1s and Cl 2p regions are acquired with pass energy of 40 eV and a 0.1 eV step size. The area examined is approximately 700 μm×300 μm for the survey scans and a 110 μm diameter spot for the high-resolution scans.

In the context of the invention, by XPS, it is possible to calculate both the extent of coating and the depth of the magnesium stearate film around the lactose particles. The extent of magnesium stearate (MgSt) coating is estimated using the following equation:

$$\% MgSt coating = ( \% {Mg}_{sample} / \% {Mg}_{ref} ) \times 100$$

where
Mgₛₐₘₚₗₑ is the amount of Mg in the analysed mixture;
Mg_{ref} is the amount of Mg in the reference sample of commercially available MgSt.

Usually the values are calculated as a mean of two different measurements. Typically, an accuracy of 10% is quoted for routinely performed XPS experiments.

Alternatively, when the excipient particles are made of lactose, preferably of alpha-lactose monohydrate, the extent of surface coating may be determined by water contact angle measurement, and then by applying the equation known in the literature as Cassie and Baxter, for example cited at page 338 of Colombo I et al Il Farmaco 1984, 39 (10), 328-341 and reported below.

$$cos {ϑ}_{mixture} = {f}_{MgSt} cos {ϑ}_{Mgst} + {f}_{lactose} cos {ϑ}_{lactose}$$

where f_{MgSt} and f_{lactore} are the surface area fractions of magnesium stearate and of lactose;
ϑ_{MgSt} is the water contact angle of magnesium stearate;
ϑ_{lactose} is the water contact angle of lactose
ϑₘᵢₓₜᵤᵣₑ are the experimental contact angle values.

For the purpose of the invention, the contact angle may be determined with methods that are essentially based on a goniometric measurement. These imply the direct observation of the angle formed between the solid substrate and the liquid under testing. It is therefore quite simple to carry out, being the only limitation related to possible bias stemming from intra-operator variability. It should be, however, underlined that this drawback can be overcome by adoption of a fully automated procedure, such as a computer assisted image analysis. A particularly useful approach is the sessile or static drop method which is typically carried out by depositing a liquid drop onto the surface of the powder in form of disc obtained by compaction (compressed powder disc method).

Within the limits of the experimental error, a good consistency has been found between the values of extent of coating as determined by XPS measurements, and those as estimated by the theoretical calculations based on the Cassie and Baxter equation.

The extent to which the magnesium stearate coats the surface of the excipient particles may also be determined by scanning electron microscopy (SEM), a well-known versatile analytical technique.

Such microscopy may be equipped with an EDX analyzer (an Electron Dispersive X-ray analyzer), that can produce an image selective to certain types of atoms, for example magnesium atoms. In this manner it is possible to obtain a clear data set on the distribution of magnesium stearate on the surface of the excipient particles.

SEM may alternatively be combined with IR or Raman spectroscopy for determining the extent of coating, according to known procedures.

In a preferred embodiment, the pharmaceutical composition filled in the multidose inhaler device comprises a fraction of fine excipients particles a) consisting of alpha-lactose monohydrate and magnesium stearate in amounts comprised between 98 and 99.0% and between 2.0 and 1.0% by weight, respectively; a fraction of coarse excipient particles b) consisting of alpha-lactose monohydrate having a particle size comprised between 210 and 360 micron, being the ratio between the fraction of fine particles a) and the fraction of coarse particles b) comprised between 1:99 and 5:95 by weight, and micronized particles of a pharmaceutically acceptable salt of tanimilast, wherein the fraction of fine particles a) is obtained by micronization for at least one hour, preferably for at least two hours.

Accordingly, the percentage of magnesium stearate in the formulation would turn out to be comprised between 0.02 and 0.1 percent by weight.

The mixing of the fraction of coarse particles b) with the fraction of fine particles a) is typically carried out in suitable mixers, e.g. tumbler mixers such as Turbula™ or Dynamix™, rotary mixers, or instant mixer such as Diosna™, for at least 5 minutes, preferably for at least 30 minutes, more preferably for at least two hours.

In a general way, the person skilled in the art shall adjust the time of mixing and the speed of rotation of the mixer to obtain a homogenous mixture.

When spheronized coarse excipient particles are desired to obtain hard-pellets according to the definition reported above, the step of mixing shall be typically carried out for at least four hours.

The active ingredients shall be present in micronized form.

Advantageously, at least 90% of all said micronized particles of the active ingredients have a volume diameter lower than 6.0 micron, preferably comprised between than 5.5 and 4.0 micron, and the volume median diameter of said particles is comprised between 1.2 and 2.5 micron, preferably between 1.3 and 2.2 micron. More advantageously, no more than 10% of all said micronized particles of the active ingredients have a diameter lower than 0.6 micron, preferably equal to or lower than 0.7 micron, more preferably equal to or lower than 0.8 micron. In a particular embodiment, no more than 10% of all said micronized particles of the active ingredients have a diameter comprised between 0.6 and 1.0 micron.

From the above particle size distribution, it follows that the width of the particle size distribution of the particles of each active ingredient, expressed as a span, should be advantageously comprised between 1.0 and 4.5, more advantageously between 1.2 and 3.0, preferably between 1.3 and 2.1, more preferably between 1.6 and 2.0. According to Chew et al J Pharm Pharmaceut Sci 2002, 5, 162-168, the span corresponds to [d(v, 0.9)−d(v,0.1)]/d(v,0.5).

Even more advantageously, at least 99% of said particles [d(v,0.99)] shall have a volume diameter equal to or lower than 7.0 micron, and substantially all the particles have a volume diameter comprised between 6.8 and 0.4 micron, preferably between 6.5 and 0.45 micron.

The size of the particles active is determined by measuring the characteristic equivalent sphere diameter, known as volume diameter, by laser diffraction. In the reported examples, the volume diameter has been determined according to European Pharmacopeia Ed. 7.0, 2.9.31, pp 295-298, using a Malvern apparatus under wet conditions, i.e. by suspending the particles in water in the presence of a surfactant.

However, other conditions such as dry conditions may be used by the skilled person in the art upon properly setting up the method.

The step of mixing the fraction of the carrier particles with all the micronized active ingredients may be carried out according to methods known in the art, for example by mixing the components in suitable known apparatus, such as a Turbula™ or Dynamix™ mixer for a sufficient period to achieve the homogeneity of the active ingredient in the final mixture.

Typically, the mixing is carried out for a time comprised between 30 and 120 minutes, preferably between 45 and 100 minutes.

The pharmaceutical composition of the invention may be suitable for delivering a therapeutic amount of all active ingredients in one or more actuations (shots or puffs) of the inhaler.

For example, the formulations will be suitable for delivering between 20 and 1000 microg per actuation, preferably between 50 and 800 microg per actuation, more preferably between 80 and 700 microg per actuation and even more preferably between 100 and 600 microg per actuation.

According to a preferred embodiment, the formulations will be suitable for delivering between 100 and 300 microg, while according to another preferred embodiment, between 200 and 800 microg, more preferably between 300 and 600 microg.

In other embodiments, the formulations will be suitable for delivering 100 microg, 200 microg 400 microg or 600 microg.

The concentration of tanimilast in the formulation could be comprised between 0.5 and 4% by weight.

Administration of the formulations of the invention is preferably indicated for the prevention and/or treatment of chronic obstructive pulmonary disease (COPD). However, said formulations might also be indicated for the prevention and/or treatment of asthma of all types and severity, including severe persistent asthma, as well as further respiratory disorders characterized by obstruction of the peripheral airways as a result of inflammation and presence of mucus such as chronic obstructive bronchiolitis.

In certain embodiments, the formulations of the invention are suitable for the prevention and/or treatment of severe and/or very severe forms of respiratory disorders, in particular severe and/or very severe forms of COPD.

The term “drug product” is to be construed to encompass the multidose dry powder inhalation device, filled with the pharmaceutical composition above described in the reservoir chamber, and molecular sieves in the desiccant chamber, and optionally a pouch which encloses the dry powder inhalation device.

As it can be appreciated from FIG. 4, the pouch contributes to reduce the absorption of moisture.

In a preferred embodiment, the pouch is a low-moisture permeable package, for example the one disclosed in EP 1760008.

The invention is illustrated in detail by the following examples.

### EXAMPLES

### Example 1—Effect of the Molecular Sieves

Eleven DPI devices as those disclosed in WO2016/000983 were filled with 0.5 g of molecular sieves (Propagroup, Italy) in the desiccant chamber and stored at 30° C. and 75% relative humidity for 21 days (PROPAGROUP). For comparison, five DPI devices filled with silica gel in the desiccant chamber were stored under the same conditions (STD).

The weight increment was determined weekly as considered representative of the absorbed humidity in the unit of time, and hence of the tendency of the device to remain more dry. The results are plotted in FIG. 3 and show that molecular sieves absorb humidity much quicker, allowing for a more dry environment to be maintained.

### Example 2—Tanimilast Dry Powder Formulation

### Process of Manufacturing

Alpha-lactose monohydrate and magnesium stearate in the ratio 98:2 w/w were weighed and mixed using the Dyna-Mix® mixer for two hours at a rotation speed of 16±1 rpm, then co-micronized using a fluid jet mill and conditioned at controlled temperature and relative humidity, leading to the pre-blend excipient preparation. Then the carrier was manufactured as follows: coarse lactose having a particle size 212-355 μm and the above pre-blend lactose/magnesium stearate were loaded into a stainless-steel container and mixed using the Dyna-Mix® mixer for 240 minutes at a rotation speed 16±1 rpm, both for 400 μg/20 mg and 800 μg/20 mg presentations.

The drug substance was, added to an aliquot of carrier and mixed for 20 minutes.

Then the pre-dispersion was mixed with the remaining carrier for further 20 minutes.

The whole product was sieved through a sieve and then final formulation mixed for further 20 minutes.

The unit formula is reported in Table 1.

TABLE 1

| | Unit formula per inhalation (μg/20 mg) | |
|---|---|---|
| Ingredients | 400 μg/20 mg | 800 μg/20 mg |
| Tanimilast | 0.400 | 0.800 |
| alpha-lactose monohydrate | 17.64 | 17.28 |
| Pre-blend (comprised of | 1.96 | 1.92 |
| lactose/magnesium stearate; 98:2 | | |
| w/w co-micronized) | | |
| alpha-lactose monohydrate | 1.9208 | 1.8816 |
| Magnesium stearate | 0.0392 | 0.0384 |
| Total | 20.000 | 20.000 |

The final formulations were filled in Nexthaler® inhaler.

The aerosol performances of the formulations at different dosages were tested using a NGI apparatus according to the procedure reported in the European Pharmacopeia Ed. 10.5, 2.9.18, p 356-360.

The following parameters were determined: i) delivered dose (DD); ii) fine particle mass (FPM); iii) fine particle fraction (FPF); iv) mass median aerodynamic diameter (MMAD); and v) geometric standard deviation (GSD).

The results in terms of mean, S.D. and R.S.D. are reported in Table 2.

TABLE 2

| | DD | FPM | FPF | MMAD | |
|---|---|---|---|---|---|
| | (μg) | (μg) | (%) | (μm) | GSD |
| | 400 μg/20 mg strength | | | | |
| Mean | 356.7 | 215.7 | 60.5 | 2.0 | 2.0 |
| S.D. | 22.94 | 15.94 | 2.73 | 0.10 | 0.05 |
| RSD % | 6.4 | 7.4 | 4.5 | 5.2 | 2.5 |
| | 800 μg/20 mg strength | | | | |
| Mean | 714.9 | 434.4 | 60.7 | 2.1 | 2.0 |
| S.D. | 41.63 | 34.82 | 2.82 | 0.09 | 0.05 |
| RSD % | 5.8 | 8.0 | 4.6 | 4.1 | 2.3 |

### Example 3—Effect of the Improvement of the Invention on the Absorption of Humidity

DPI devices as those disclosed in WO 2016/000983, separately incorporating the modifications of the invention were stored at 35° C. and 75% relative humidity for at least 14 days. The weight increment was determined weekly as considered representative of the absorbed humidity in the unit of time, and hence of the tendency of the device to remain more dry. The results are plotted in FIG. 4, which shows a differential weight over time due to moisture absorption of inhalers incorporating different separate embodiments of the invention compared to a standard inhaler. As it can be appreciated, all the improvements of the invention are effective in decreasing the absorption of moisture.

1.-14. (canceled)

15. A drug product comprising a multidose dry powder inhalation device having a pharmaceutical composition within,
the multidose dry powder inhalation device comprising a medicament chamber and a desiccant chamber adjacent to the medicament chamber and filled with molecular sieves, and
the pharmaceutical composition comprising tanimilast as active ingredient, together with an excipient.

16. The drug product according to claim 15, wherein the molecular sieves are made of alkaline salts of aluminosilicates, with pores having a diameter from 2 to 50 Angstrom.

17. The drug product according to claim 16, wherein the pores have a diameter from 3 to 20 Angstrom.

18. The drug product according to claim 15, wherein the multidose dry powder inhalation device comprises:
a casing having a mouthpiece and delimiting an inhalation channel connected to an opening of the mouthpiece;
a container for storing a powdered medicament, the container being placed in the casing;
a dispensing device placed in the casing and configured to dispense unit doses of the powdered medicament from the container to the inhalation channel for inhalation through the mouthpiece; and
a cover engageable with the casing to close the mouthpiece, the cover comprising a sealing element for humidity resistance,
wherein, when the cover is engaged with the casing and closes the mouthpiece, a main portion of the sealing element is coupled to the opening to tight close said opening.

19. The drug product according to claim 18, wherein the sealing element has a hardness between 10 Shore A and 60 Shore A.

20. The drug product according to claim 19, wherein the sealing element has a hardness between 20 Shore A and 40 Shore A.

21. The drug product according to claim 20, wherein the sealing element has a hardness between 25 Shore A and 35 Shore A.

22. The drug product according to claim 18, wherein the sealing element comprises or is made of silicone.

23. The drug product according to claim 18, wherein the sealing element is over-molded to the cover or push-fitted in the cover and optionally glued to the cover.

24. The drug product according to claim 15, wherein the pharmaceutical composition comprises the active ingredient in micronized form.

25. The drug product, according to claim 15, wherein the excipient comprises:
a fraction of fine excipient particles consisting of a physiologically acceptable excipient and magnesium stearate; and
a fraction of coarse excipient particles made of a physiologically acceptable excipient and having a particle size comprised between 200 and 400 micron.

26. The drug product according to claim 25, wherein the fraction of fine excipient particles is obtained by micronization for at least 2 hours.

27. The drug product according to claim 25, wherein the physiologically acceptable excipient is alpha-lactose monohydrate.

28. The drug product according to claim 15, wherein the excipient comprises
a fraction of fine excipient particles consisting of alpha-lactose monohydrate and magnesium stearate in amounts comprised between 98 and 99.0% and between 2.0 and 1.0% by weight, respectively; and
a fraction of coarse excipient particles consisting of alpha-lactose monohydrate having a particle size comprised between 210 and 360 micron,
wherein a ratio between the fraction of fine excipient particles and the fraction of coarse excipient particles is comprised between 1:99 and 5:95 by weight,
and wherein tanimilast is in form of micronized particles of a pharmaceutically acceptable salt of tanimilast.

29. The drug product according to claim 28, wherein the fraction of fine excipient particles is obtained by micronization for at least one hour.

30. The drug product according to claim 29, wherein the fraction of fine excipient particles is obtained by micronization for at least two hours.

31. A process for manufacturing the drug product according to claim 15, said process comprising
filling the medicament chamber of a multidose dry powder inhalation device with the pharmaceutical composition comprising a pharmaceutically acceptable salt of tanimilast, and
filling the desiccant chamber of said device with the molecular sieves.
